(19) United States
(12) Patent Application Publication
(10) Pub. No.: US 2025/0114054 A1
(43) Pub. Date: Apr. 10, 2025

(54) DETECTION OF JAW ARCH GEOMETRY

(71) Applicant: DÜRR DENTAL SE, Bietigheim-Bissingen (DE)

(72) Inventors: Robert WENZKO, Ingersheim (DE); Maximilian BEMMANN, Leipzig (DE)

(73) Assignee: DÜRR DENTAL SE, Bietigheim-Bissingen (DE)

(21) Appl. No.: 18/908,695

(22) Filed: Oct. 7, 2024

(30) Foreign Application Priority Data
Oct. 9, 2023 (DE) 102023127425.2

(51) Int. Cl. A61B 6/04; A61C 5/00

(52) U.S. Cl. CPC A61B 6/04; A61C 5/007

(57) ABSTRACT

The invention relates to a device and a method for determining information about at least a portion of a dental arch.

### CROSS-REFERENCE TO RELATED APPLICATIONS

This application claims priority under 35 U.S.C. 119 to German Patent Application No. 102023127425.2, filed Oct. 9, 2023; the disclosure of which is incorporated herein by reference in its entirety.

### FIELD

The present invention relates to a device and a method for determining information about at least a portion of a dental arch.

### BACKGROUND

Patient positioning is particularly important in medical imaging systems for dental technology. In today's systems, patient positioning is carried out by (dental) medical professionals. Positioning is therefore naturally prone to errors and is subject to error. As a result, images taken by a medical imaging system often have to be taken again or the images are of insufficient quality and have to be processed afterwards, which is time-consuming.

EP 4 129 191 A1 relates to a method for producing digital panoramic images using extraoral X-ray apparatuses using prior knowledge of the patient's anatomy in order to avoid downstream software solutions with complex reconstruction. Existing image material, such as one or more previous panoramic images or 3D images or one or more optical 3D scans of the jaw arch shape, is used as model-based prior knowledge of the patient's anatomy. In other words, the trajectory is not recalculated, but rather calculated in advance using a type of artificial intelligence (AI) from existing material in order to follow the most accurate trajectory possible for each patient.

In EP 3 688 719 A1, an individually adapted path of an X-ray source is defined using a digital 3D surface representation of at least a portion of a patient's teeth.

US 2022/0192616 A1 discloses a reduction of artifacts in the image by means of three panoramic images, wherein the first panoramic image forms the basis, the second panoramic image weights the artifacts, and the third represents the result of the subtracted artifacts from the second image in the first image.

US 2010/0055634 A1 discloses a bite plate with an intraoral vibration plate.

EP 2 932 903 A1 discloses a craniostat for patient positioning with a forehead rest.

Furthermore, WO 2023/083648 A1 relates to a method for improving a medical scan of a patient's head anatomy. The method comprises providing a plurality of scan datasets related to a head anatomy. The method comprises providing intraoral data related to one or more intraoral features of the patient. The method comprises determining, using the intraoral data, those parts of the scan dataset during acquisition of which a movement related to the intraoral features was detected. The method comprises constructing and/or correcting the medical scan, from the scan dataset, in response to the determination.

Even in prior art, which reduces or even eliminates manual interventions by staff during patient positioning, the necessary effort is quite high, for example due to preliminary calculations, subsequent calculations and/or additional images.

There is therefore a need for an improved device for determining information about at least a portion of a dental arch and for an associated method.

### SUMMARY

According to a first aspect of the invention, a device for determining information about at least a portion of a dental arch is proposed. The device includes a bite block assembly and an evaluation unit. The bite block assembly includes at least one pressure sensor. The bite block assembly is designed to be received between at least a portion of a row of teeth of an upper jaw and at least a portion of a row of teeth of a lower jaw. The at least one pressure sensor is designed to sense pressure information about pressure and/or pressure exerted by at least the portion of the row of teeth of the upper jaw and/or by at least the portion of the row of teeth of the lower jaw on the at least one pressure sensor. The evaluation unit is designed to determine information about at least a portion of a dental arch based on the pressure information sensed by the at least one pressure sensor.

The determination of at least the portion of the dental arch by means of pressure information sensed by at least one pressure sensor present in a bite block assembly allows an accurate yet simple determination.

Normally, the cord-like assembly of the teeth in the upper and lower jaw may be referred to as a row of teeth. The upper and lower rows of teeth in an adult normally consist of 16 teeth each. In children, the upper and lower rows of teeth normally consist of 10 teeth each when fully developed.

The dental arch (also called the alveolar arch) in humans usually has the shape of a parabola with the vertex between teeth no. 11 and 21 in the upper jaw (corresponding to teeth no. 31 and 41 in the lower jaw). The parabolic/horseshoe-shaped row of teeth in the upper jaw may be referred to as a dental arch. The parabolic/horseshoe-shaped row of teeth in the lower jaw and the upper jaw may be referred to as a dental arch. Consequently, it may be said that the term row of teeth refers to the series of teeth, while the term dental arch defines a geometry and/or a shape beyond the mere series of teeth. Therefore, it may also be said that the bite block assembly can be designed to be received between at least a portion of a dental arch of an upper jaw and at least a portion of a dental arch of a lower jaw. The dental arch may also be referred to as jaw arch.

The information about at least the portion of the dental arch can include information about at least a portion of a dental arch curve or a dental arch shape. The information about at least a portion of the dental arch may comprise information about at least one tooth of the dental arch, for example information about exactly one tooth. It is also conceivable that the information about at least a portion of the dental arch includes information about at least a part or portion of a tooth of the dental arch.

The bite block assembly may include an occlusal splint or be designed as an occlusal splint. In a specific example, the bite block assembly may include an occlusal splint and a shaft connected directly or indirectly to the occlusal splint. In other words, the occlusal splint may be designed as one piece with the shaft to form the bite block assembly. The occlusal splint may merge directly into the shaft or at least one further portion may be provided between the occlusal splint and the shaft. The bite block assembly may be connected or connectable wirelessly and/or by wire to the evaluation unit, i.e. the connection between the bite block assembly and the evaluation unit may include a wired connection, a wireless connection or a combination of both. The bite block assembly can use the wireless and/or wired connection to transmit pressure information sensed by the at least one pressure sensor to the evaluation unit. The pressure information can include information about a force exerted or acting on the at least one pressure sensor by at least the portion of the row of teeth of the upper jaw and/or by at least the portion of the row of teeth of the lower jaw. The pressure information may therefore also be referred to as force information.

For sensing pressure information about pressure exerted by at least the portion of the row of teeth of the upper jaw, the at least one pressure sensor can, for example, be arranged on a side of the bite block assembly facing the upper jaw or can be actuatable via a side of the bite block assembly facing the upper jaw. For sensing pressure information about pressure exerted by at least the portion of the row of teeth of the lower jaw, the at least one pressure sensor can, for example, be arranged on a side of the bite block assembly facing the lower jaw or can be actuatable via a side of the bite block assembly facing the lower jaw. For sensing pressure information about pressure exerted by at least the portion of the row of teeth of the upper jaw and pressure information about pressure exerted by at least the portion of the row of teeth of the upper jaw, the at least one pressure sensor can, for example, be arranged on a side of the bite block assembly facing the upper jaw or on a side of the bite block assembly facing the lower jaw, or can be actuatable via a side of the bite block assembly facing the upper jaw and a side of the bite block assembly facing the lower jaw.

The evaluation unit can be designed to determine, as the information about at least the portion of the dental arch, information about at least a portion of a dental arch of the upper jaw. Additionally, or alternatively, the evaluation unit can be designed to determine, as the information about at least the portion of the dental arch, information about at least a portion of a dental arch of the lower jaw. In other words, the evaluation unit can be designed to determine, as the information about at least the portion of the dental arch, only information about at least a portion of a dental arch of the upper jaw, or to determine, as the information about at least the portion of the dental arch, only information about at least a portion of a dental arch of the lower jaw, or to determine, as the information about at least the portion of the dental arch, both information about at least a portion of a dental arch of the lower jaw and information about at least portion of a dental arch of the upper jaw. The information about at least a portion of a dental arch of the lower jaw may comprise information about at least a portion or part of a tooth of the lower jaw or about several teeth of the lower jaw. The information about at least a portion of a dental arch of the upper jaw may comprise information about at least a portion or part of a tooth of the upper jaw or about several teeth of the upper jaw.

The evaluation unit can be designed to determine, as the information about at least the portion of the dental arch, information about the entire dental arch of the upper jaw. Additionally, or alternatively, the evaluation unit can be designed to determine, as the information about at least the portion of the dental arch, information about the entire dental arch of the lower jaw.

The evaluation unit can be designed to determine, as the information about at least the portion of the dental arch, information about at least a portion of a dental arch averaged from the dental arch of the upper jaw and the dental arch of the lower jaw. The averaged dental arch can be determined in various ways from the dental arch of the upper jaw and the dental arch of the lower jaw. For example, in a simple design, the average value can simply be formed from the information about at least the portion of the dental arch of the upper jaw and the information about at least the portion of the dental arch of the lower jaw in order to determine the information about at least the portion of the averaged dental arch. Alternatively, the information about at least the portion of the dental arch of the upper jaw and the information about at least the portion of the dental arch of the lower jaw can be combined with one another according to a predetermined weighting or a predetermined weighting scheme in order to determine the information about at least the portion of the averaged dental arch. The evaluation unit can be designed to determine, as the information about at least the portion of the dental arch, information about the entire averaged dental arch.

The evaluation unit can be designed to determine information about geometry and/or a width and/or a position of a dental arch, for example a dental arch of the upper jaw and/or a dental arch of the lower jaw, based on the pressure information sensed by the at least one pressure sensor.

The evaluation unit can be designed to determine information about a width and/or a position of a jaw including the upper jaw and the lower jaw, based on the pressure information sensed by the at least one pressure sensor. Additionally, or alternatively, the evaluation unit can be designed to determine information about a width and/or a position of the upper jaw, based on the pressure information sensed by the at least one pressure sensor. Additionally, or alternatively, the evaluation unit can be designed to determine information about a width and/or a position of the lower jaw, based on the pressure information sensed by the at least one pressure sensor.

The at least one pressure sensor can be designed and arranged to sense, as the pressure information, information about one or more bite points in the row of teeth of the upper jaw and/or in the row of teeth of the lower jaw. The at least one pressure sensor can be designed and arranged to sense, as the pressure information, only information about one or more bite points in the row of teeth of the upper jaw. For this purpose, the at least one pressure sensor can be arranged on a side of the bite block assembly facing the upper jaw or can be actuatable via a side of the bite block assembly facing the upper jaw. The at least one pressure sensor can be designed and arranged to sense, as the pressure information, only information about one or more bite points in the row of teeth of the lower jaw. For this purpose, the at least one pressure sensor can be arranged on a side of the bite block assembly facing the lower jaw or can be actuatable via a side of the bite block assembly facing the lower jaw. The at least one pressure sensor can be designed and arranged to sense, as the pressure information, both information about one or more bite points in the row of teeth of the upper jaw and information about one or more bite points in the row of teeth of the lower jaw. For this purpose, the at least one pressure sensor can be arranged on a side of the bite block assembly facing the upper jaw and on a side of the bite block assembly facing the lower jaw. Additionally, or alternatively, the at least one pressure sensor can be actuatable via a side of the bite block assembly facing the upper jaw and via a side of the bite block assembly facing the lower jaw.

The determination of bite points by means of at least one pressure sensor in the bite assembly allows a particularly accurate and yet simple determination of information about at least the portion of the dental arch.

The at least one pressure sensor can be designed and arranged to sense, as the pressure information, information about buccal and/or palatal positions of one or more tooth cusps and/or of one or more cutting surfaces of teeth of the row of teeth of the upper jaw and/or of teeth of the row of teeth of the lower jaw. In this case, buccal is normally the technical term in dentistry for tooth surfaces facing the cheeks, i.e. for the outside of the teeth. Furthermore, palatal is normally the technical term in dentistry for tooth surfaces facing the palate, i.e. for the inside of the teeth. Additionally, or alternatively, the at least one pressure sensor can be designed and arranged to sense, as the pressure information, information about one or more tooth misalignments and/or an oblique bite of one or more teeth in the row of teeth of the upper jaw and/or of one or more teeth in the row of teeth of the lower jaw. The one or more tooth misalignments can, for example, be derivable directly from the pressure information, for example a pressure bite.

The at least one pressure sensor can include a sensor array or sensor field, in particular a sensor array or sensor field with individual sensors and/or thin film sensors and/or foil sensors. In a specific design, the at least one pressure sensor can be designed as a sensor array or sensor field, in particular as a sensor array or sensor field with individual sensors and/or thin film sensors and/or foil sensors.

The evaluation unit can be designed to determine information about a trajectory of a medical imaging apparatus, in particular an extraoral X-ray apparatus, based on the information about at least the portion of the dental arch. The extraoral X-ray apparatus can be designed as a panoramic X-ray apparatus. The X-ray apparatus can include an X-ray emitter and an X-ray detector. The X-ray emitter and/or the X-ray detector can move on a trajectory to be determined, in particular move around a part, for example the head, of a patient. A standard trajectory can be stored or saved in the medical imaging apparatus, which is followed independently of patient-specific information. Based on the information about at least the portion of the dental arch, a patient-specific trajectory of the medical imaging apparatus can be determined. For example, the standard trajectory can be adapted based on the information about at least the portion of the dental arch in order to determine the patient-specific trajectory. The adaptation can be carried out taking into account deviations between a standard dental arch and the information about at least the portion of the dental arch. The standard dental arch can be stored or saved in the medical imaging apparatus. In general, the evaluation unit can be designed to completely determine a patient-specific trajectory of a medical imaging apparatus based on the information about at least the portion of the dental arch. Alternatively, the evaluation unit can be designed to adapt one or more parameters of an existing trajectory, based on the information about at least the portion of the dental arch, for example to adapt a contour and/or a position of the trajectory in space, in order to determine the patient-specific trajectory of the medical imaging apparatus.

The determination of the information about a trajectory of a medical imaging apparatus based on the information about at least the portion of the dental arch allows an accurate and patient-specific determination of the information about the trajectory. In particular, a determination of the information about the trajectory can be made possible without or at least almost without human interaction when positioning a patient.

The evaluation unit can be designed to determine information about a sagittal plane, based on the pressure information sensed by the at least one pressure sensor. The sagittal plane is a plane that normally runs vertically parallel to the sutura sagittalis (sagittal suture of the skull).

The evaluation unit can be further designed to adapt information about a/the trajectory of a medical imaging apparatus, based on the information determined about the sagittal plane. This allows the patient-specific trajectory to be adapted even better and/or more accurately to the patient.

The bite block assembly can further include at least one measuring system. The measuring system can include at least one strain gauge on an upper side of a shaft of the bite block assembly and/or at least one strain gauge on an underside of the shaft of the bite block assembly. In other words, the measuring system can include only at least one strain gauge on an upper side of a shaft of the bite block assembly. Alternatively, the measuring system can only include at least one strain gauge on an underside of the shaft of the bite block assembly. Alternatively, the measuring system can include both at least one strain gauge on an upper side of a shaft of the bite block assembly and at least one strain gauge on an underside of the shaft of the bite block assembly. In a specific example, the measuring system can be designed as at least one strain gauge on an upper side of a shaft of the bite block assembly and/or as at least one strain gauge on an underside of the shaft of the bite block assembly. The assembly of strain gauges on the upper side and/or the underside of the shaft can result in a wave-like shape (waveform) of the shaft. Strain gauges (SGs) are normally understood to be measuring systems for sensing stretching and/or compressing deformations. SGs change their electrical resistance even with small deformations and can be employed as strain sensors. Applied to the bite block assembly, they can sense when the bite block assembly deforms ever so slightly. This deformation (stretching) then leads to a change in the resistance of the SG. The change in the resistance of the SG can be evaluated by the evaluation unit. The bite block assembly can be arranged at an angle in space, at least in portions. For example, at least a portion of the bite block assembly can be inclined or run at an angle with respect to an occlusal plane and/or with respect to a horizontal.

The evaluation unit can be designed to determine information about an inclination of an occlusal plane, based on information sensed by the at least one measuring system. If, for example, strain gauges on the top and bottom of the shaft of the bite block assembly output/deliver the same values, no force is exerted on the shaft by the patient. This means that the occlusal plane is also in the correct position. In other words, if no force is exerted on the shaft by the patient, the bite block assembly including the bite splint and the shaft represents the perfect position, e.g. inclination and rotation, of the occlusal plane. If an uneven distribution of force between the top and bottom of the shaft is detected, the position of the occlusal plane can be adapted accordingly. The plane on which the teeth of the upper and lower jaw meet is normally referred to as the occlusal plane. The information determined about the occlusal plane can be used by the evaluation unit to adapt the information about at least the portion of the dental arch. The information determined about the occlusal plane can be used by the evaluation unit to adapt the information about the trajectory of the medical imaging apparatus.

According to a second aspect, a method for determining information about at least a portion of a dental arch is proposed. The method comprises providing a bite block assembly with at least one pressure sensor. The method further comprises receiving the bite block assembly between at least a portion of a row of teeth of an upper jaw and at least a portion of a row of teeth of a lower jaw. The method further comprises sensing pressure information about pressure exerted by at least the portion of the row of teeth of the upper jaw and/or by at least the portion of the row of teeth of the lower jaw on the at least one pressure sensor of the bite block assembly. The method further comprises determining, by an evaluation unit, information about at least a portion of a dental arch, based on the pressure information sensed by the at least one pressure sensor.

A third aspect relates to a computer program with program code means which, when loaded into a computer or a processor (for example a microprocessor, microcontroller or digital signal processor (DSP)) or executed on a computer or processor (e. g., a microprocessor, microcontroller or DSP), causes the computer or processor (e. g., microprocessor, microcontroller or DSP) to carry out one or more steps or all steps of the method steps previously described with respect to the evaluation unit. In addition, a program storage medium or computer program product is provided with the computer program mentioned.

Furthermore, for example, the computer program according to the third aspect can be stored in the evaluation unit of the device according to the first aspect and cause the evaluation unit to carry out one or more or all of the aspects and/or steps of the method according to the second aspect described above with respect to the evaluation unit. Furthermore, for example, the computer program according to the third aspect can be stored in the evaluation unit of the device according to the first aspect and cause the evaluation unit to carry out one or more or all of the aspects and/or features described above with respect to the evaluation unit.

A fourth aspect relates to a medical imaging apparatus, for example an X-ray apparatus, in particular a panoramic X-ray apparatus, with the device described herein according to the first aspect and/or with the evaluation unit described with respect to the device according to the first aspect.

Even if some of the aspects described above were described with respect to the evaluation unit, these aspects can also be implemented in a corresponding manner in the medical imaging apparatus, the method or a computer program implementing the method or the computing unit. Likewise, the aspects described above with respect to the methods can be implemented in the medical imaging apparatus, the method or a computer program implementing the method or the computing unit.

### BRIEF DESCRIPTION OF THE DRAWINGS

The present disclosure will be further explained with reference to figures. In these figures, schematically:

FIG. 1 shows a skull with a bite splint;

FIG. 2 shows a device according to an exemplary embodiment with a bite block assembly and an evaluation unit;

FIG. 3 shows a flow chart of a method according to an exemplary embodiment;

FIG. 4 shows bite points of a row of teeth determined by the device from FIG. 2 as an example;

FIG. 5 shows a dental arch curve determined by means of the device from FIG. 2 as an example;

FIG. 6 shows possible adaptations of the dental arch from FIG. 5 according to a width of the jaw;

FIG. 7 shows an example of a rotational adaptation of the dental arch curve determined as an example in FIG. 6; and

FIG. 8 shows a variant of the device from FIG. 2 with strain gauges.

### DETAILED DESCRIPTION

Specific details are set forth below, without limitation thereto, in order to provide a thorough understanding of the present disclosure. However, it will be apparent to one skilled in the art that the present disclosure may be used in other exemplary embodiments that may differ from the details set forth below. For example, specific configurations and designs of an evaluation unit and/or a bite block assembly are described, which are not to be regarded as limiting.

Those skilled in the art appreciate that the explanations set forth below can be implemented using hardware circuits, software means or a combination thereof. The software means can be associated with programmed microprocessors, an artificial intelligence or a general computer, an ASIC (Application Specific Integrated Circuit) and/or DSPs (Digital Signal Processors). Furthermore, it should be understood that even if the details below are described with respect to a method, these details can also be implemented in a suitable device unit, a computer processor or a memory connected to a processor, the memory being provided with one or more programs that carry out the method when executed by the processor.

The exemplary embodiments can be implemented and/or used in various medical imaging apparatus. Merely by way of example, X-ray apparatuses, in particular panoramic X-ray apparatuses, may be mentioned here.

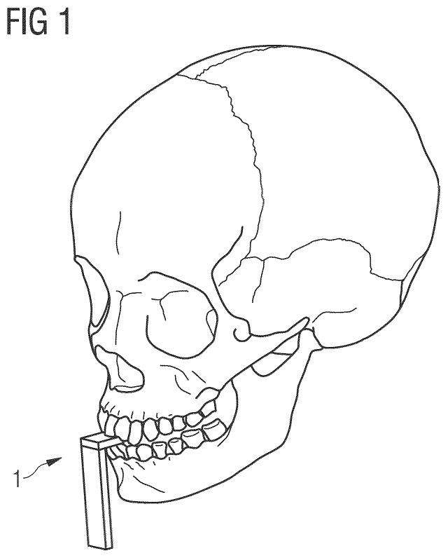

FIG. 1 shows schematically a patient's skull and a conventional bite splint 1. As can be seen, the patient bites into bite splint 1 before and/or during X-ray imaging(s). The hope is that the teeth will engage with the bite splint to partially fixate the head and, in particular, that the patient's head will remain as still as possible and in a fixed, e. g. calibrated, position during X-ray imaging(s). In particular, the bite block is used to limit or at least almost prevent translation of the skull. In this way, the hope is to be able to produce X-ray images that are as accurate and sharp as possible.

In practice, however, it turns out that this procedure is prone to errors. On the one hand, the head usually does not remain in the desired resting position. On the other, the positioning is based on the experience of the practice staff and is therefore subject to human error and misjudgment.

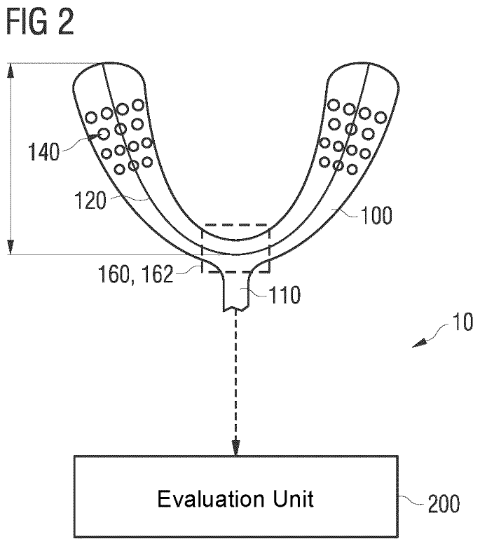

FIG. 2 shows a schematic representation of a device 10 according to an exemplary embodiment. Device 10 serves to determine information about at least a portion of a dental arch. Device 10 includes a bite block assembly 100 and an evaluation unit 200. Bite block assembly 100 includes at least one pressure sensor 140. In the exemplary design according to FIG. 2, the at least one pressure sensor 140 is designed as a plurality of pressure sensors 140 by way of example. In other words, bite block assembly 100 includes a plurality of pressure sensors 140 by way of example. The exemplary design of the bite block assembly 100 from FIG. 2 includes a bite splint 120 and a shaft 110. Bite splint 120 is, by way of example, the part of bite block assembly 100 that is received or placed between the teeth of a patient. In the exemplary design shown in FIG. 2, several pressure sensors 140 of the plurality of pressure sensors are arranged on one side/at one end (left in FIG. 2) and several pressure sensors 140 of the plurality of pressure sensors are arranged on another side/at another end (right in FIG. 2). In FIG. 2, the number of pressure sensors on one side corresponds, by way of example and without being limited thereto, to the number of pressure sensors on the other side. The pressure sensors can be arranged so as to be distributed over bite splint 120. They can be triggerable from one side (top or bottom) or from both sides (top and bottom) of bite splint 120.

Bite block assembly 100, in particular bite splint 120 in FIG. 2, is designed to be received between at least a portion of a row of teeth of an upper jaw and at least a portion of a row of teeth of a lower jaw. The plurality of pressure sensors 140 from FIG. 2, as an example of at least one pressure sensor 140, is designed to sense pressure information about pressure exerted on the plurality of pressure sensors 140 by at least the portion of the row of teeth of the upper jaw and/or by at least the portion of the row of teeth of the lower jaw. Evaluation unit 200 is designed to determine information about at least a portion of a dental arch, based on at least a subset of the pressure information sensed by the plurality of pressure sensors 140. In order to transmit the pressure information sensed by pressure sensors 140 to the evaluation unit, pressure sensors 140 can be connected wirelessly and/or by wire to evaluation unit 200.

Bite splint 120 can have a dental arch shape or a partial dental arch shape. Bite splint 120 can include at least one sensor array as pressure sensors 140. For example, bite splint 120 can include a first sensor array on one side (top side) and a second sensor array on the other side (bottom side). The at least one sensor array can form a surface for measuring the jaw geometry. For example, a first sensor array can form a surface for measuring the upper jaw geometry at/on bite splint 120, and a second sensor array can form a surface for measuring the lower jaw geometry at/on bite splint 120. For this purpose, individual sensors or foil sensors can be used. Evaluation unit 200 can then use this data to determine a patient-specific jaw arch, for example an averaged one. Using the jaw arch, evaluation unit 200 can determine/calculate/choose a patient-specific apparatus movement curve that is at least (almost) perfectly fitting. Furthermore, a layer position (a sharp area) can be adapted in a patient-specific manner, for example in a panoramic image or in a 3D model creation. The quality of the raw image data is thus so high that post-processing of the image can be omitted or reduced to a minimum.

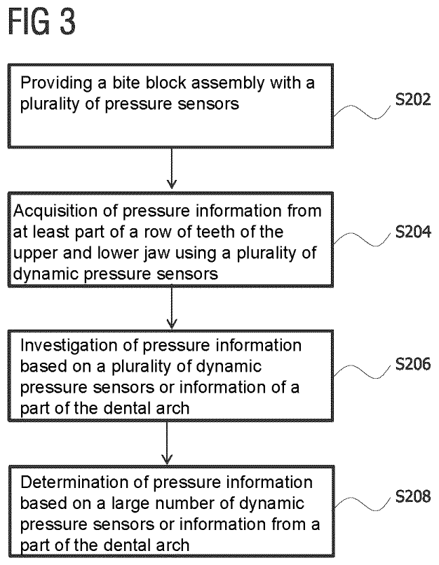

FIG. 3 shows a flow chart of a method for determining information about at least a portion of a dental arch, which can be carried out in or by device 100 from FIG. 1. In step S202, a bite block assembly 100 is provided with at least one pressure sensor 140, and with reference to the design from FIG. 2 with a plurality of pressure sensors 140. In step S204, bite block assembly 100, and with reference to the design from FIG. 2 at least a portion of bite splint 120, is received or placed between at least a portion of a row of teeth of an upper jaw and at least a portion of a row of teeth of a lower jaw. In step S206, pressure information about the pressure exerted by at least the portion of the row of teeth of the upper jaw and/or by at least the portion of the row of teeth of the lower jaw on the plurality of pressure sensors 140 of bite block assembly 100, and more specifically with reference to FIG. 2, bite splint 120, is sensed. In step S208, information about at least a portion of a dental arch is determined by evaluation unit 200, based on the pressure information sensed by the plurality of pressure sensors 140.

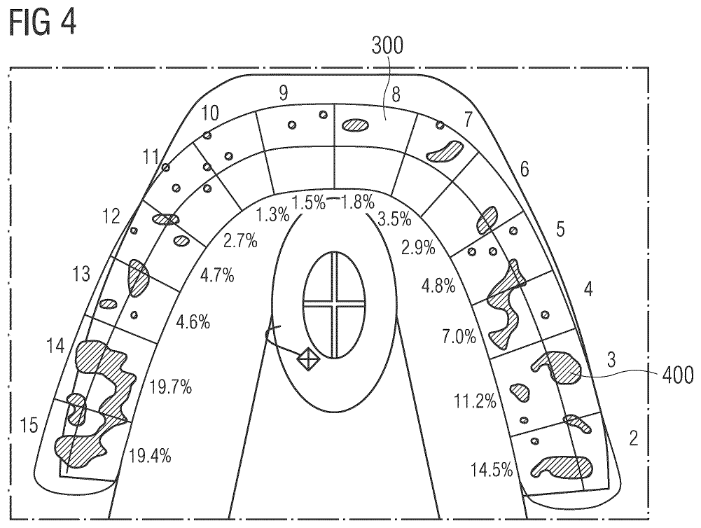

Further details of device 100 from FIG. 2 and the method from FIG. 3 will now be described with reference to FIGS. 4 to 8. Even if in FIGS. 4 to 8 reference is made only to the upper jaw by way of example, the explanations also apply accordingly to the lower jaw.

FIG. 4 shows a representation of bite points/bite areas 400 of a row of teeth 300 of the upper jaw. For the sake of simplicity, the term bite points 400 will be used consistently below, even if they are areas in some instances and could therefore also be referred to as bite areas 400. Pressure sensors 140 sense pressure exerted by row of teeth 300 of the upper jaw on pressure sensors 140 of bite splint 120. The corresponding pressure information is transmitted to evaluation unit 200, for example queried by evaluation unit 200 or transmitted to evaluation unit 200 directly after sensing by pressure sensors 140. The pressure information received by evaluation unit 200 can be presented as bite points 400 of tooth row 300 of the upper jaw, as shown by way of example in FIG. 4.

More specifically, bite points 400 of the respective individual teeth of row of teeth 300 of the upper jaw and the row of teeth of the lower jaw are sensed/measured by means of pressure sensors 140. From bite points 400, evaluation unit 200 can determine, for example, buccal and/or palatal positions of individual teeth, in particular individual tooth cusps and/or cutting surfaces. Additionally, or alternatively, evaluation unit 200 can determine, for example, one or more tooth misalignments and/or an oblique bite from bite points 400. Evaluation unit 200 can, for example, derive the one or more tooth misalignments directly from bite points 400.

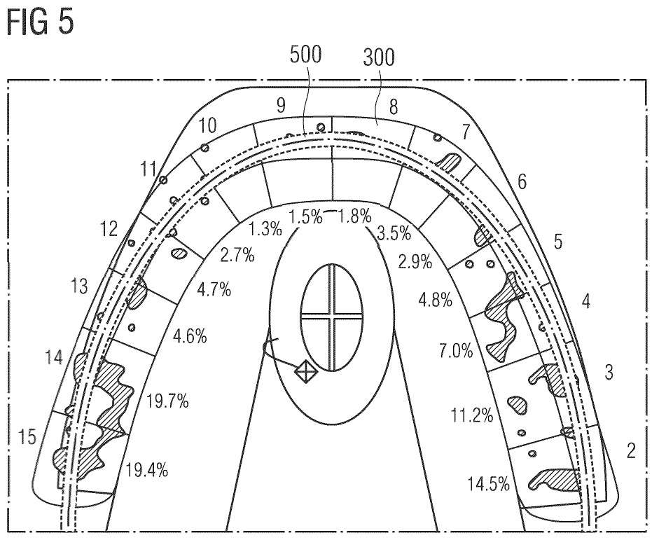

Evaluation unit 200 evaluates bite points 400 and creates information about a patient-specific dental arch from bite points 400, which is illustrated in FIG. 5 as a dental arch curve 500 along row of teeth 300. This dental arch curve 500 is illustrated by way of example in FIG. 5 and can differ from the form shown by way of example. The dental arch (also called the alveolar arch) in humans usually has the shape of a parabola with the vertex between teeth no. 11 and 21 in the upper jaw (corresponding to teeth no. 31 and 41 in the lower jaw). The upper jaw dental arch is normally somewhat larger than that of the lower jaw. Dental arch curve 500 is determined by evaluation unit 200 taking into account bite points 400.

According to the above description with respect to the upper jaw, the dental arch curve of the lower jaw can also be determined in a corresponding manner by evaluation unit 200. By combining the data from the upper and lower jaw, a parabolic, patient-specific tooth curve/dental arch curve that is as accurate as possible can be determined.

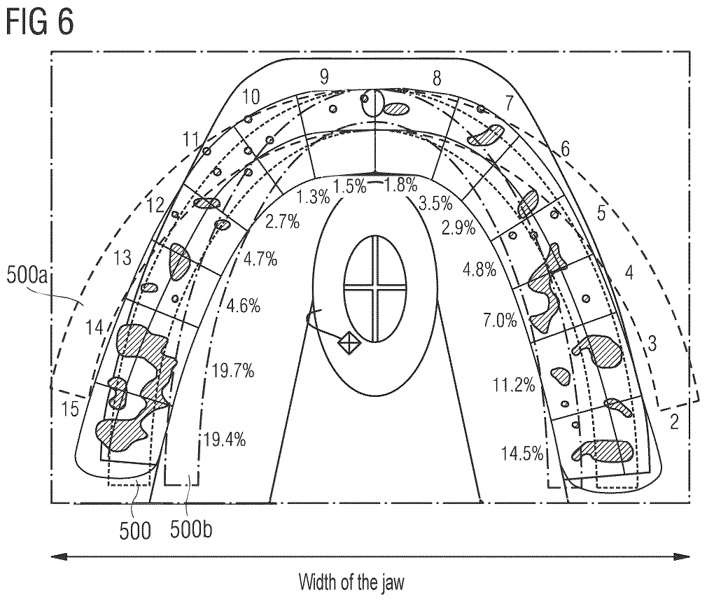

FIG. 6 shows schematically how evaluation unit 200 can adapt determined dental arch curve 500. The determined dental arch curve can first be supplemented by a so-called “region of interest (ROI)”, i.e. an area of interest. The ROI can be located around dental arch curve 500. The ROI is illustrated by the wider area around dental arch curve 500 in FIG. 6. For example, the dental arch curve can be in the middle of the ROI. The thickness of the ROI increases in the area of the jaw arch from the front (front teeth) to the back (molars) and is therefore different in the area of the jaw arch (front teeth=thin ROI->molars=thick ROI), as illustrated in FIG. 6. To achieve the best possible image quality, the ROI should always be chosen only as thick or wide as necessary and therefore not be unnecessarily large or inaccurate. In FIG. 6 and in the figures thereafter, the ROI can already be seen when dental arch curve 500 of the upper jaw is shown. This is not to be understood as limiting, but merely serves as an illustration. This means that an ROI can be determined for dental arch curve 500 of the upper jaw and the dental arch curve of the lower jaw and for the combined patient-specific dental arch curve. Alternatively, the ROI of the dental arch curves for the upper and lower jaw can be omitted and instead the ROI can be determined only for the combined patient-specific dental arch curve.

Evaluation unit 200 can adapt determined dental arch curve 500 based on a position and/or width of the jaw, in particular in the present case based on a position and/or width of the upper jaw, for example. To this end, evaluation unit 200 determines the position and/or width of the jaw, based on the example described the position and/or width of the upper jaw. This determination can be made, for example, taking into account determined bite points 400. By way of example, referring to the determined width of the jaw, evaluation unit 200 adapts dental arch curve 500 taking into account the determined width. In the case of a very wide upper jaw, dental arch curve 500 can, for example, be adapted in such a way that, for example, a wide dental arch curve 500a is obtained as a result. In the case of a very narrow upper jaw, dental arch curve 500 can, for example, be adapted in such a way that, for example, a narrow dental arch curve 500b is obtained as a result. For example, it is assumed here that dental arch curve 500 is not adapted/changed or only minimally so that dental arch curve 500 shown is obtained as a result after adaptation. The adaptation of the dental arch curve 500 can also be referred to as curve fit.

In summary, dental arch curve 500 can be adapted taking into account the position and width of the jaw, for example the upper jaw. The adaptation can be carried out on the dental arch curve of the upper jaw and/or on the dental arch curve of the lower jaw and/or on the combined patient-specific dental arch curve.

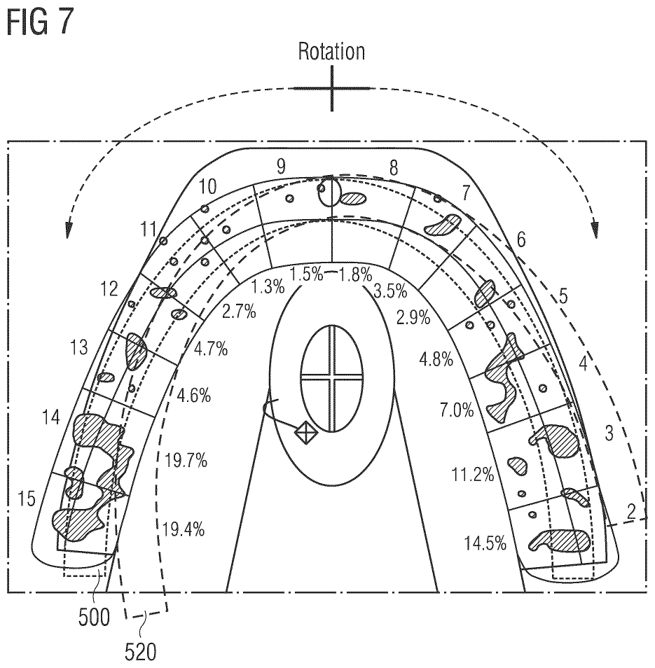

FIG. 7 shows a schematic representation of how evaluation unit 200 can take rotation in the horizontal plane into account. To this end, evaluation unit 200 can determine a horizontal plane based on determined bite points 400. By means of a corresponding rotation in the horizontal plane, dental arch curve 500 can be brought into the correct orientation. For example, the occlusal plane can be brought as close as possible to the horizontal plane or can correspond to the horizontal plane. By way of example, FIG. 7 shows how dental arch curve 500 can be converted into a rotated dental arch curve 520 by rotation. For example, parabolic dental arch curve 500, or more specifically, the entire ROI around dental arch curve 500, can be rotated around a vertex (between teeth 11; 21 and 31; 41) of the parabola in the occlusal plane into rotated dental arch curve 520 including its ROI.

Curve fitting can therefore measure and/or take into account a rotation in the horizontal plane. This means that dental arch curve 500 can be adapted automatically.

The patient's sagittal plane can also be determined using a suitably large pressure sensor surface. If the actual sagittal plane is known, a rotation of dental arch curve 500 can be calculated. For example, as shown in FIG. 7, dental arch curve 500 can be converted into rotated dental arch curve 520.

An apparatus movement curve, for example a panoramic recording curve of an X-ray apparatus, can be determined based on determined patient-specific dental arch curve 500. For example, a rotation start point can be adapted. Furthermore, by adapting the apparatus movement curve, a ratio of a distance between the X-ray emitter and the patient/object can be adapted to a distance between the patient/object and the X-ray detector.

The patient's sagittal plane can also be determined using a sufficiently large pressure sensor surface. If the actual sagittal plane is known, the apparatus movement curve can also be adapted.

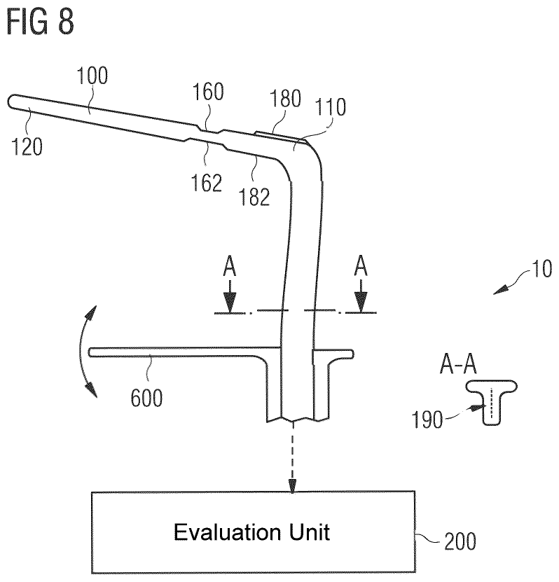

FIG. 8 shows a representation of a variant of device 10 from FIG. 1 in a side view.

Bite block assembly 100 according to this variant includes an upper notch 160 (see also FIG. 2) for the front incisors (teeth no. 11, 21) of the upper jaw and a lower notch 162 (see also FIG. 2) for the front incisors (teeth no. 31, 41) of the lower jaw. The incisors of the upper jaw can engage in notch 160 and the incisors of the lower jaw can engage in notch 162. Furthermore, strain gauges (SGs) 180, 182 are arranged on shaft 110 of bite block assembly 100. More specifically, an upper SG 180 is arranged or applied on an upper side of shaft 110, and a lower SG 182 is arranged or applied on the underside of shaft 110. By means of SGs 180, 182, a force applied on SG 180 by the upper jaw and a force applied on SG 182 by the lower jaw can be determined. If the forces applied are the same, an inclination of an occlusal plane can remain unchanged in a neutral position. If the force applied by the upper jaw is greater than that of the lower jaw, the inclination of the occlusal plane can be adapted accordingly by evaluation unit 200, for example, it can be inclined differently according to the difference.

This means the inclination of the occlusal plane can be determined by the force applied on shaft area 110 of bite splint 120. If SGs 180, 182 on the top and bottom of shaft 110 provide the same values, no force is exerted on shaft 110 by the patient. Since bite splint 120 is arranged at the correct angle for the image position, the occlusal plane is also in the correct position. Consequently, the occlusal plane can be inclined based on the determined inclination. Rotations on the occlusal plane can thus be detected and the position of the patient can be corrected (=feedback system) and/or the incorrect positioning can be included in the trajectory calculation, for example the panoramic recording curve. As shown in FIG. 8, bite block assembly 100, or alternatively bite splint 110 of bite block assembly 100, does not run parallel to a horizontal, but rather inclined to a horizontal. Furthermore, bite block assembly 100, or alternatively bite splint 110 of bite block assembly 100, normally runs obliquely or inclined to an occlusal plane. In addition, bite block assembly 100 or shaft 110 of bite block assembly 100 is slightly wavy in the side view shown in FIG. 8 due to notches 160, 162 and SGs 180, 182.

As outlined above, the inclination of the occlusal plane can be adapted. This means that rotations on the occlusal plane can be detected, and the position of the patient can be corrected (=feedback system) and/or incorrect positioning can be included in the trajectory calculation, for example the panoramic image curve. For example, with a panoramic image it is recommended that the patient's spine is slightly overextended in order to be able to display the front teeth area with as little artifacts as possible. Furthermore, the applied X-ray dose is usually increased when imaging the front teeth area because this is where the rays have to travel the longest distance through the tissue and therefore collect the most artifact information. The patient's head should therefore ideally be inclined slightly downwards. This can be achieved indirectly by ensuring that the Frankfurt horizontal and the apparatus horizontal are congruent. The angle between the Frankfurt horizontal and the occlusal plane is known and is approximately 5.6°. The correct position of the planes can thus be ensured or established by measurement. This means that the spine can be assumed to be correctly hyperextended and the use of a Frankfurt laser in the apparatus can be omitted. Optionally, the X-ray dose can also be reduced, as it is possible to prevent radiation from passing through a non-hyperextended spine.

In general, when imaging a tooth or several teeth, it is possible to reduce, for example, minimize, a so-called field of view (FOV) of an imaging system. In this way, a radiation dose can be reduced, for example, minimized. In addition, an improved, for example the best possible, image quality can be achieved. For example, it is possible to focus the field of view on a single tooth. This tooth can be imaged from the tip to the root, for example.

In addition, an inclination of chin rest 600 can be sensed and measured. The determined inclination of the chin rest can be taken into account in the evaluations by evaluation unit 600. In this way, a compression and/or expansion of shaft 110 of bite block assembly 100 can be sensed.

In FIG. 8, a section A-A is also shown. In the corresponding section figure, a line 190, for example a copper line, can be seen. The sensed pressure information/pressure signals of pressure sensors 140 140 can be routed to evaluation unit 200 via line 190, or to a unit from which the pressure information of pressure sensors 140 can be transmitted wirelessly or by wire to evaluation unit 200.

By means of the device and the method described, a jaw geometry can be determined at least almost exactly.

The totality of the measurement data in conjunction with the movement capabilities of the imaging system enables correction of positioning errors that may be caused by the user or the patient.

The following advantages are also achieved. By measuring the jaw arch, an automatic selection of the jaw width and/or an option for adapting a patient-specific trajectory/a trajectory for each patient, in particular a panoramic curve, can be achieved. There is little or no interaction during positioning and therefore less potential for error. Possible positioning errors are compensated for and thus the movement of a C-arm is adapted (starting point for translation and rotation). Better positioning can be achieved by integrating a feedback system. The measurement data can be saved for evaluation of the jaw geometry. Better positioning for a digital volume tomography (DVT) volume is achieved.

Overall, image quality and user-friendliness are improved.

In contrast to the prior art, neither a pre-calculation nor a post-calculation of the dental arch is necessary, but rather a dental arch curve taken directly by the patient. A complex preliminary creation/establishing (in an extra work step) of the jaw geometry or consideration of existing, sometimes incorrect or poor quality image material can therefore be omitted without replacement.

This means that post-calculations, post-processing, positioning lasers, incorrect positioning by personnel and/or additional recordings (scout) can be omitted.

1. A device for determining information about at least a portion of a dental arch, comprising:
a bite block assembly with at least one pressure sensor, wherein the bite block assembly is configured to be received between at least a portion of a row of teeth of an upper jaw and at least a portion of a row of teeth of a lower jaw, and wherein the at least one pressure sensor is configured to sense pressure information about pressure exerted by at least the portion of the row of teeth of the upper jaw and/or by at least the portion of the row of teeth of the lower jaw on the at least one pressure sensor; and
an evaluation unit which is configured to determine arch information about at least a portion of a dental arch based on the pressure information sensed by the at least one pressure sensor.

2. The device according to claim 1, wherein the evaluation unit is configured to determine, as the arch information about at least the portion of the dental arch, information about at least a portion of a dental arch of the upper jaw and/or information about at least a portion of a dental arch of the lower jaw.

3. The device according to claim 1, wherein the evaluation unit is configured to determine, as the arch information about at least the portion of the dental arch, information about at least a portion of a dental arch averaged from information about at least a portion of a dental arch of the upper jaw and information about at least a portion of a dental arch of the lower jaw.

4. The device according to claim 1, wherein the evaluation unit is configured to determine arch information about a width and/or a position of a jaw including the upper jaw and the lower jaw, based on the pressure information sensed by the at least one pressure sensor.

5. The device according to claim 1, wherein the at least one pressure sensor is configured and arranged to sense, as the pressure information, information about one or more bite points in the row of teeth of the upper jaw and/or in the row of teeth of the lower jaw.

6. The device according to claim 1, wherein the at least one pressure sensor is configured and arranged to sense, as the pressure information, information about buccal and/or palatal positions of one or more tooth cusps and/or of one or more cutting surfaces of teeth of the row of teeth of the upper jaw and/or of teeth of the row of teeth of the lower jaw.

7. The device according to claim 1, wherein the at least one pressure sensor includes a sensor array with individual sensors and/or thin-film sensors and/or foil sensors or is configured as a sensor array which includes individual sensors and/or thin-film sensors and/or foil sensors.

8. The device according to claim 1, wherein the evaluation unit is configured to determine apparatus information about a trajectory of a medical imaging apparatus based on the arch information about at least the portion of the dental arch.

9. The device according to claim 1, wherein the evaluation unit is configured to determine information about a sagittal plane, based on the pressure information sensed by the at least one pressure sensor.

10. The device according to claim 9, wherein the evaluation unit is configured to adapt information about a trajectory of a medical imaging apparatus based on the information determined about the sagittal plane.

11. The device according to claim 1, wherein the bite block assembly further comprises at least one measuring system or at least one strain gauge on an upper side of a shaft of the bite block assembly and/or at least one strain gauge on an underside of the shaft of the bite block assembly.

12. The device according to claim 11, wherein the evaluation unit is configured to determine information about an inclination of an occlusal plane, based on information sensed by the at least one measuring system.

13. The device according to claim 12, wherein the evaluation unit is configured to adapt information about a trajectory of a medical imaging apparatus based on the information determined about the inclination of the occlusal plane.

14. A method for determining information about at least a portion of a dental arch, comprising:
providing a bite block assembly with at least one pressure sensor;
receiving the bite assembly between at least a portion of a row of teeth of an upper jaw and/or at least a portion of a row of teeth of a lower jaw;
sensing pressure information about pressure exerted by at least the portion of the row of teeth of the upper jaw and/or by at least the portion of the row of teeth of the lower jaw on the at least one pressure sensor of the bite block assembly; and
determining, by an evaluation unit, arch information about at least a portion of a dental arch, based on the pressure information sensed by the at least one pressure sensor.

15. The device according to claim 8, wherein the medical imaging apparatus is a medical X-ray apparatus.
